(19) United States
(12) Patent Application Publication
(10) Pub. No.: US 2023/0225402 A1
(43) Pub. Date: Jul. 20, 2023

(54) LEAK-PROOF ATOMIZER AND INHALATION DEVICE

(71) Applicant: SHENZHEN ZUN YI PIN TECHNOLOGY CO., LTD., Shenzhen (CN)

(72) Inventors: MIAOWEN YANG, Shenzhen (CN); YONGHUI LIN, Shenzhen (CN); SHIMEI PAN, Shenzhen (CN); HUIFU SHUAI, Shenzhen (CN); QIANG XUE, Shenzhen (CN); SHAOQING YAN, Shenzhen (CN)

(21) Appl. No.: 17/711,245

(22) Filed: Apr. 1, 2022

Related U.S. Application Data
(63) Continuation-in-part of application No. PCT/CN2022/073048, filed on Jan. 20, 2022.

(51) Int. Cl. A24F 40/42; A24F 40/10; A24F 40/44; A24F 40/485

(52) U.S. Cl. CPC A24F 40/42; A24F 40/10; A24F 40/44; A24F 40/485; A61M 11/02

(57) ABSTRACT

An atomizer, producing an inhalable vapor, includes a liquid storage chamber, an air duct, and an atomizing core in the liquid storage chamber. The atomizing core includes a core shell and a liquid guiding cotton. The core shell includes a base and a housing. A sealing member is disposed between the air duct and the core shell, the sealing member includes a sealing body and a first connecting pipe extending from the sealing body facing the air duct. An elastic part is disposed on an outer wall of the first connecting pipe, the air duct abuts the sealing body, a bottom of the sealing body abuts the liquid guiding cotton, the first connecting pipe is located in the air duct, and the elastic part presses tightly against an inner wall of the air duct to provide sealing of liquid in all circumstances. An inhalation device is also disclosed.

### FIELD

The subject matter relates to the field of loading an atomizable substance, after being atomized, into a human body, and in particular to an atomizer and an inhalation device including the atomizer.

### BACKGROUND

An inhalation device generally includes an atomizer and a power supply assembly. The atomizer can heat a liquid atomizable substance to generate smoke or aerosol by means of atomization, for a user to inhale. The power supply assembly is used to supply power to the atomizer. The atomizer includes a pipe for conveying the smoke, a liquid storage chamber for storing the liquid atomizable substance, and an atomizing core for heating the liquid atomizable substance.

However, when the inhalation device is being carried, the liquid atomizable substance in the liquid storage chamber may leak and flow into a space between the atomizing core and the pipe due to poor sealing between the pipe and the atomizing core, thereby affecting the taste experienced by the user.

### BRIEF DESCRIPTION OF THE DRAWINGS

FIG. 1 is a perspective view of an atomizer according to an embodiment of the present disclosure.

FIG. 2 is an exploded view of the atomizer of FIG. 1.

FIG. 3 is an exploded view of the atomizer of FIG. 1 from another perspective.

FIG. 4 is an exploded view of an atomizing core of the atomizer of FIG. 1.

FIG. 5 is a cross-sectional view along line V-V of FIG. 1.

FIG. 6 is a perspective view of an inhalation device according to an embodiment of the present disclosure.

FIG. 7 is an exploded view of the inhalation device of FIG. 6.

FIG. 8 is an exploded view of part of the inhalation device of FIG. 6 from another perspective.

FIG. 9 is a cross-sectional view along line IX-IX of FIG. 6.

FIG. 10 is an exploded view of a liquid guiding cotton and a second oil stopper of an atomizer according to a second embodiment of the present disclosure.

### DETAILED DESCRIPTION

The following clearly describes the technical solutions in the embodiments of this disclosure. The described embodiments are merely some but not all of the embodiments of this disclosure. Unless otherwise defined, all technical and scientific terms used herein have the same meanings as usually understood by a person skilled in the technical field of this disclosure. The terms used in the specification of this disclosure herein are merely intended for describing specific embodiments but are not intended to limit this disclosure.

Hereinafter, embodiments of the present disclosure will be described in detail. The present disclosure may be embodied in many different forms and should not be construed as limited to the exemplary embodiments set forth herein. Rather, these exemplary embodiments are provided for ease of understanding by those of ordinary skill in the art.

The term “comprising,” when utilized, means “including, but not necessarily limited to”; it specifically indicates open-ended inclusion or membership in the so-described combination, group, series, and the like.

### First Embodiment

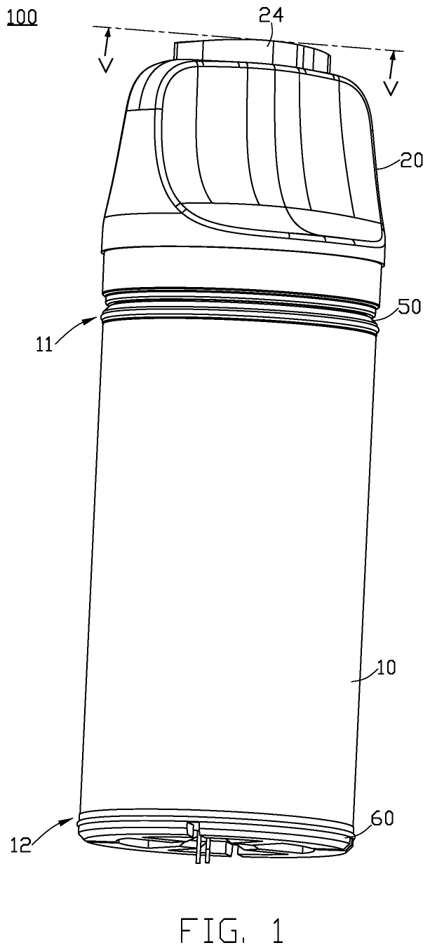

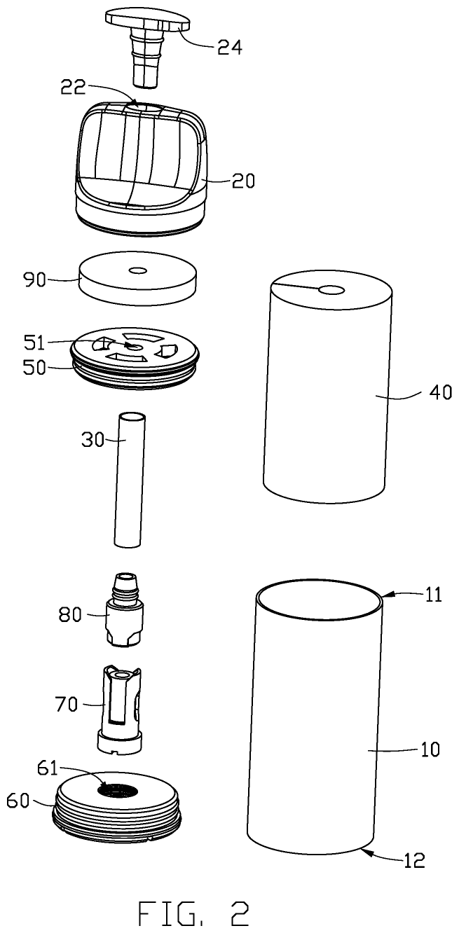

Referring to FIGS. 1 and 2, an atomizer 100 includes a liquid storage chamber 10, a suction part 20, an air duct 30, a liquid storage cotton 40, a first oil stopper 50, a second oil stopper 60, and an atomizing core 70. The liquid storage chamber 10 includes a first end 11 and a second end 12 opposite to the first end 11, and the suction part 20 is disposed on the first end 11 of the liquid storage chamber 10. The first oil stopper 50 is received in the first end 11 of the liquid storage chamber 10 and is located between the suction part 20 and the liquid storage chamber 10. The first oil stopper 50 has a first vent 51. The second oil stopper 60 seals the second end 12 and the second oil stopper 60 has a second vent 61. The air duct 30 is accommodated in the liquid storage chamber 10 and communicates with the first vent 51 and the second vent 61 through the air duct 30. The atomizing core 70 is arranged between the air duct 30 and the second oil stopper 60. The liquid storage cotton 40 is arranged in the liquid storage chamber 10, disposed annularly on outer walls of the air duct 30 and the atomizing core 70. The liquid storage cotton 40 is used to store a liquid atomizable substance, which includes an active ingredient. The first oil stopper 50 and the second oil stopper 60 are used to seal the liquid atomizable substance in the liquid storage cotton 40 in the liquid storage chamber 10. The atomizing core 70 is used to heat and atomize the liquid atomizable substance in the liquid storage cotton 40 to obtain an aerosol. The air duct 30 is used to guide the aerosol to the suction part 20 for a user to inhale.

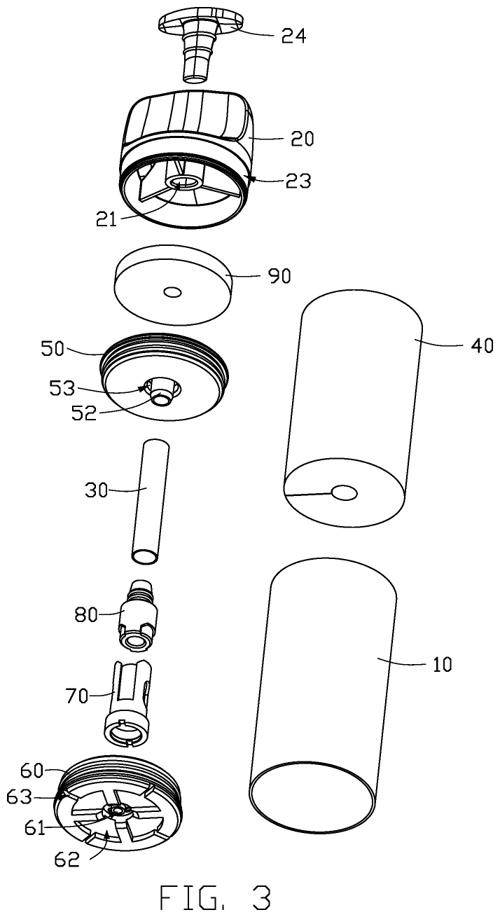

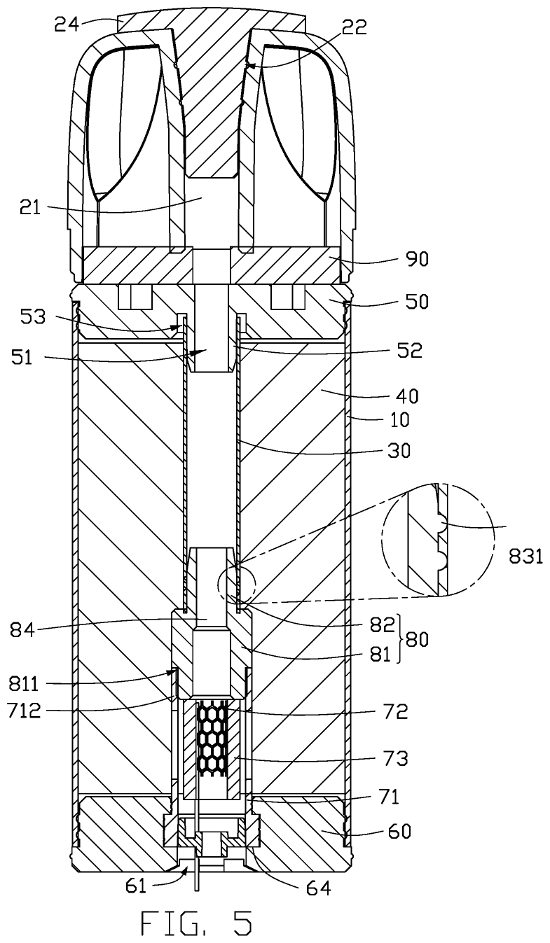

Referring to FIGS. 3 and 5, a second connecting pipe 52 extends from a surface of the first oil stopper 50 facing the second end 12. The second connecting pipe 52 communicates with the first vent 51, and their central axes are collinear. At the connection between the second connecting pipe 52 and the first oil stopper 50, a recess 53 is formed, which extends around a periphery of the second connecting pipe 52. One end of the air duct 30 abuts against the recess 53, and the second connecting pipe 52 extends into the air duct 30. An outer wall of the second connecting pipe 52 abuts against an inner wall of the air duct 30, so that the second connecting pipe 52 tightly cooperates with the air duct 30, preventing the liquid atomizable substance from flowing into the gap between the air duct 30 and the first oil stopper 50, which affects the taste experienced by the user.

Referring to FIGS. 2 and 3, in some embodiments, the liquid storage cotton 40 is an integrated structure. The liquid storage cotton 40 can store a large amount of the liquid atomizable substance, and the liquid atomizable substance is stably absorbed in the liquid storage cotton 40, reducing the fluidity of the liquid atomizable substance in the liquid storage chamber 10, and thereby reducing the risk of leakage and improving the safety of carrying and the service life of the atomizer 100.

Referring to FIGS. 3 and 5, in some embodiments, the suction part 20 includes a suction port 22 and an airflow channel 21 communicating with the suction port 22. The suction port 22 communicates with the air duct 30 through the airflow channel 21. The air duct 30, the suction port 22, the airflow channel 21, and the liquid storage chamber 10 may be arranged coaxially to facilitate egress of the aerosol generated by the atomizing core 70, guiding it along the airflow channel 21 to the suction port 22, for the user to inhale. In some embodiments, the shape of the suction part 20 may be ergonomically made, such as round, oval, or duck-billed.

In some embodiments, the suction part 20 may also be provided with a dust plug 24. The dust plug 24 is detachably arranged in the suction port 22 to prevent external dust or water vapor from entering into the atomizer 100 when the atomizer 100 is not in use.

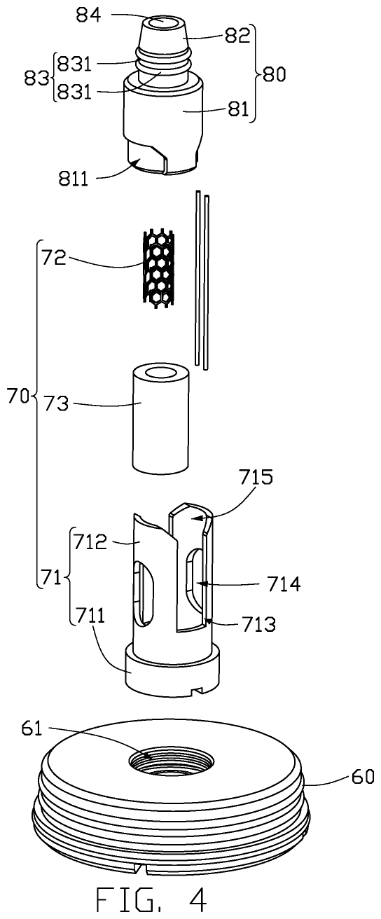

Referring to FIGS. 4 and 5, the atomizing core 70 includes a core shell 71, a heating element 72, and a liquid guiding cotton 73. The core shell 71 includes a base 711 and a housing 712 arranged on the base 711. A cross section of the core shell 71 is approximately circular and has a holding cavity 715. The holding cavity 715 communicates with the base 711 and the housing 712. The housing 712 is also provided with two through slots 713 communicating with the holding cavity 715. Along a direction from the first oil stopper 50 to the second oil stopper 60, the two through slots 713 symmetrically divide the housing 712 into two parts. The liquid guiding cotton 73 is an integrated structure, and a cross section of the liquid guiding cotton 73 is substantially a ring in shape. In some embodiments, the housing 712 is also provided with a liquid inlet 714. The liquid atomizable substance in the liquid storage cotton 40 is transported to the liquid guiding cotton 73 through the through slots 713 and the liquid inlet 714, so as to improve the uniformity of the liquid atomizable substance absorbed by the liquid guiding cotton 73.

The heating element 72 is arranged at a center of the liquid guiding cotton 73, and the liquid guiding cotton 73 equipped with the heating element 72 is accommodated in the core shell 71. The base 711 abuts against the second oil stopper 60. The second vent 61 of the second oil stopper 60 is configured to communicate with an external environment. The center of the liquid guiding cotton 73 corresponds in position to a center of the second vent 61.

Referring to FIGS. 4 and 5, in some embodiments, the heating element 72 may be a heating element which is helical or net-like in shape. A heating part of the heating element 72 is close to an inner wall of the liquid guiding cotton 73 so that the heating element 72 can fully atomize the liquid atomizable substance in the liquid guiding cotton 73.

In some embodiments, a sealing member 80 is disposed between the air duct 30 and the core shell 71 to seal a connecting location between the air duct 30 and the core shell 71 to prevent the liquid atomizable substance in the liquid storage cotton 40 from flowing into the air duct 30 through a gap between the air duct 30 and the core shell 71. The sealing member 80 includes a connecting hole 84 which communicates with the air duct 30 and the liquid guiding cotton 73. The aerosol generated by the atomization of the heating element 72 flows out from the center of the liquid guiding cotton 73, the connecting hole 84, the air duct 30, and the suction port 22 for the user to inhale.

Referring to FIGS. 4 and 5, the sealing member 80 includes a sealing body 81 and a first connecting pipe 82 extending from a surface of the sealing body 81. The first connecting pipe 82 faces the air duct 30, the air duct 30 abuts against the sealing body 81, and the first connecting pipe 82 extends into the inner wall of the air duct 30, thereby increasing an area of contact between the first connecting pipe 82 and the air duct 30 and reducing the leakage of the liquid atomizable substance from the liquid storage cotton 40 to the air duct 30.

In some embodiments, in order to further improve the sealing between the first connecting pipe 82 and the air duct 30, an elastic part 83 is disposed on an outer wall of the first connecting pipe 82. The elastic part 83 includes a plurality of elastic rings 831, and each elastic ring 831 is compressed against the air duct 30. This arrangement not only avoids loosening of the sealing member 80 from the air duct 30, but also prevents the liquid atomizable substance infiltrating into the gap between the sealing member 80 and the air duct 30 from infiltrating into the air duct 30.

In some embodiments, part of an outer wall of the sealing body 81 is concave to form a groove 811 corresponding to a top structure of the housing 712. Part of the housing 712 is accommodated in the groove 811, and a bottom of the sealing body 81 abuts against the liquid guiding cotton 73, thereby increasing an area of contact between the housing 712 and the sealing body 81, improving the connection stability of the sealing body 81 to the housing 712.

Referring to FIGS. 3 and 5, in some embodiments, a first liquid absorbent cotton 90 is arranged between the suction part 20 and the first oil stopper 50, and the first liquid absorbent cotton 90 is used to absorb any liquid atomizable substance overflowing from the liquid storage chamber 10, thereby reducing the risk of leakage.

Referring to FIGS. 3 and 5, in some embodiments, a part of an inner wall of the second vent 61 extends inward to form a boss 64, and the base 711 abuts against the boss 64. A plurality of air passages 63 are arranged on a surface of the second oil stopper 60 away from the air duct 30, the air passages 63 communicate with the second vent 61. An external air flow enters the second vent 61 through the air passages 63 and carries the aerosol generated by the heating element 72 to flow to the suction port 22 for the user to inhale.

In some embodiments, the surface of the second oil stopper 60 away from the air duct 30 defines a plurality of air grooves 62. Each of the air grooves 62 communicates with the air passage 63 and the second vent 61, so that the air flow through the air passage 63 is directed to the second vent 61 through the air grooves 62. In some embodiments, there are four air passages 63, the number of the air grooves 62 is the same as that of the air passages 63, and the shape of the air grooves 62 is substantially fan-shaped.

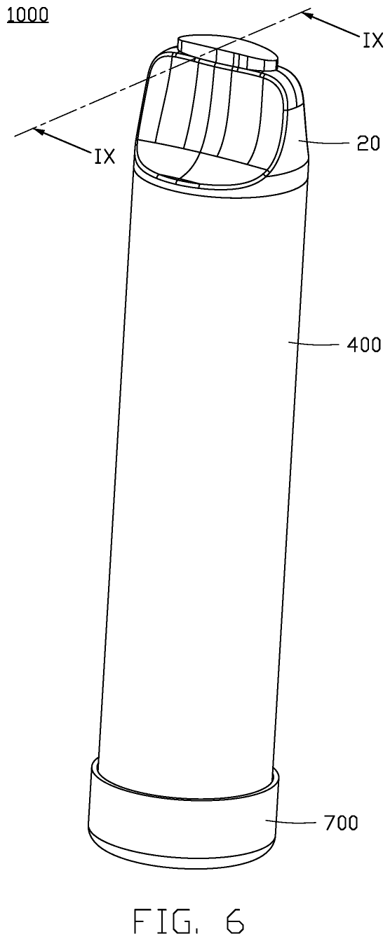

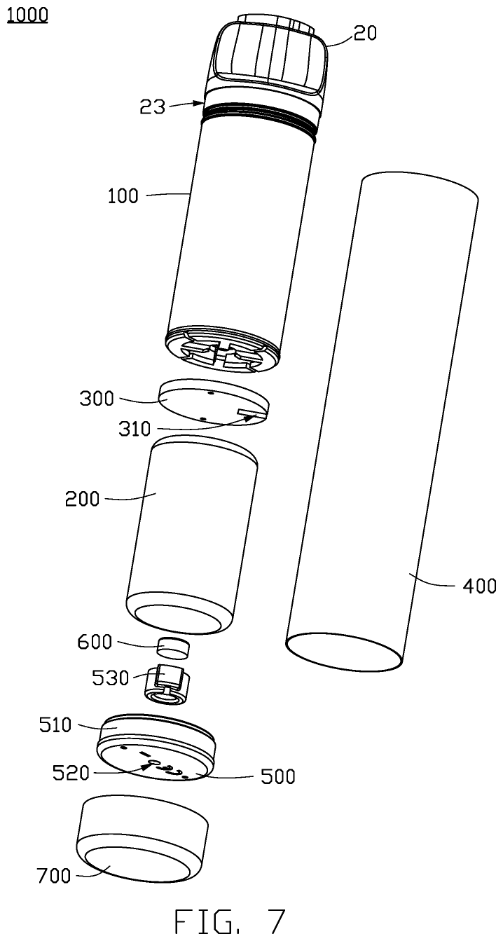

Referring to FIGS. 6 and 7, an inhalation device 1000 includes a casing 400, a power supply assembly 200, a second liquid absorbent cotton 300, and the atomizer 100. The atomizer 100, the power supply assembly 200, and the second liquid absorbent cotton 300 are all accommodated in the casing 400.

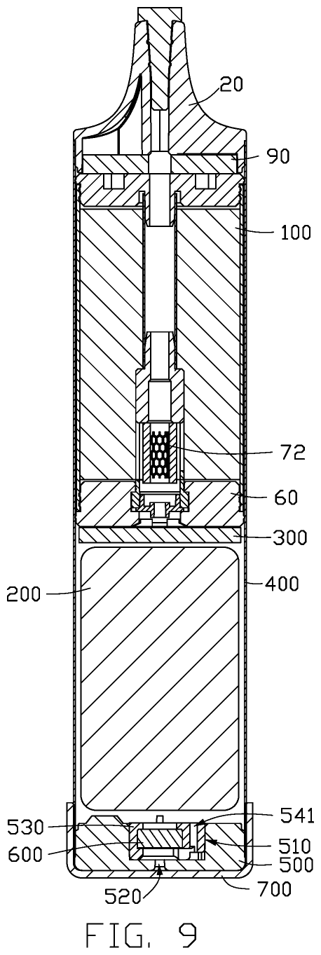

Referring to FIGS. 7 and 9, the suction part 20 of the atomizer 100 extends out of one end of the casing 400. An outer wall of the suction part 20 defines a clamping groove 23 extending around a periphery of the suction part 20, and an upper end of the casing 400 is fixed in the clamping groove 23 by an adhesive (not shown). In other embodiments, the casing 400 and the suction part 20 may be fixed to each other by means of magnetism or interference fit. The power supply assembly 200 may be a cylindrical battery. The second liquid absorbent cotton 300 is arranged between the second oil stopper 60 and the power supply assembly 200. The second liquid absorbent cotton 300 is used to adsorb the liquid atomizable substance which overflows at the liquid storage cotton 40 or at the liquid guiding cotton 73, to reduce the risk of leakage and the risk of liquid dripping on the power supply assembly 200.

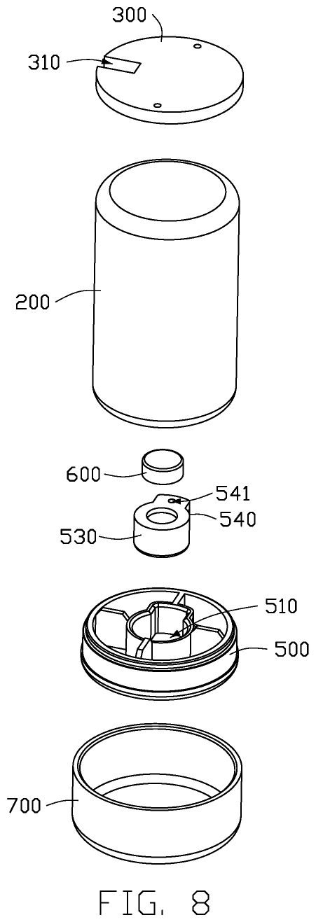

Referring to FIGS. 7 and 8, in some embodiments, the second liquid absorbent cotton 300 includes a notch 310. In this case, an installation position of the second liquid absorbent cotton 300 can be easily determined when assembling the inhalation device 1000, so that the atomizer 100 and the power supply assembly 200 can be quickly assembled. In addition, the external air flow can flow into the air grooves 62 (shown in FIG. 3) through the notch 310, and then flow into the second vent 61.

In some embodiments, the inhalation device 1000 may be an electronic cigarette, that is, the liquid atomizable substance is e-liquid. In other embodiments, the inhalation device 1000 is not limited to an electronic cigarette, but may also be other devices for atomizing the liquid atomizable substance into aerosol for the user to inhale. For example, the inhalation device 1000 may be an instrument conveying the aerosol formed by atomizing, such as a medical atomization inhaler for treating upper respiratory tract diseases. In this case, the liquid atomizable substance is a liquid drug for treating upper respiratory tract diseases. A patient can inhale drug mist, formed by atomizing the drug liquid, into the respiratory tract and the alveoli through breathing, thus carrying out local drug treatment of the upper respiratory tract. The inhalation device 1000 can be used to convey the aerosol formed by atomizing the liquid atomizable substance onto surface of the skin. For example, the aerosol is applied onto the skin for cosmetic purposes by the inhalation device 1000.

Compared with intravenous injection and oral administration, a manner of atomizing drug liquid into aerosol allows the aerosol to directly act on a required treatment location, achieving better treatment effect. In an intravenous injection, the drug liquid is injected into the vein through a syringe and finally transported to the required treatment location. The drug liquid goes through blood circulation in the intravenous injection process. In the oral administration by swallowing, the drug liquid passes through the digestive system and goes through blood circulation to reach the required treatment location. The two manners of intravenous injection and oral administration cannot make the drug liquid fully act on the required treatment location. In addition, on some occasions, compared with the oral administration, the manner of atomizing drug liquid is more convenient.

Referring to FIGS. 7, 8, and 9, in some embodiments, the inhalation device 1000 may also include a mount 500 and an air flow sensor 600. The air flow sensor 600 and the mount 500 are arranged in the casing 400, the mount 500 is arranged at an end of the power supply assembly 200 away from the atomizer 100 and covers an end of the casing 400. The mount 500 includes an air inlet 520. One side of the mount 500 facing the power supply assembly 200 defines a positioning groove 510 communicating with the air inlet 520. A positioning ring 530 is disposed in the positioning groove 510. The air flow sensor 600 is accommodated in the positioning groove 510 and the positioning ring 530 corresponds in position to the air inlet 520. A protrusion 540 is disposed on an outer wall of the positioning ring 530. The protrusion 540 includes an air hole 541 communicating with an inner cavity of the positioning ring 530. When in use, the external air flow enters the mount 500 through the air inlet 520 and passes through the air flow sensor 600, the air flow sensor 600 detects and starts transmitting information to a controller (not shown), and the controller controls the power supply assembly 200 to supply power to the heating element 72. Next, the air flow flows into the air passages 63 through the air hole 541 and the gap between the casing 400 and the power supply assembly 200 (shown in FIG. 3). And then, the aerosol formed by heating and atomizing the liquid atomizable substance by the heating element 72 passes through the air duct 30 and the suction port 22 with the air flow for the user to inhale. In some embodiments, the air flow sensor 600 is a microphone.

A cover 700 is detachably sleeved on an end of the casing 400 away from the suction part 20. When not in use, the cover 700 covers the end of the casing 400 to prevent external impurities or water vapor from entering the casing 400 through the air inlet 520 of the mount 500, thereby protecting the power supply assembly 200 in the casing 400.

In the atomizer 100, the first connecting pipe 82 of the sealing member 80 extends into the air duct 30 and the elastic part 83 abuts and presses against the inner wall of the air duct 30, so that the sealing member 80 is sealed on the air duct 30, thereby preventing the liquid atomizable substance stored in the liquid storage chamber 10 from flowing into the air duct 30 through the gap between the air duct 30 and the sealing member 80. The taste of the aerosol is not spoiled, a service life of the atomizer 100 is improved, and loosening of the sealing member 80 in the air duct 30 when carried does not take place.

### Second Embodiment

A difference between the second embodiment and first embodiment is the structure of the atomizer 100. The difference is as follows.

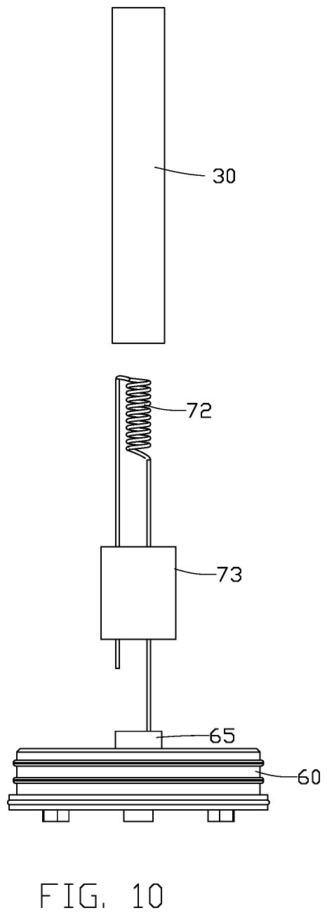

Referring to FIGS. 5 and 10, a bulge loop 65 is disposed on a side of the second oil stopper 60 facing the liquid guiding cotton 73, and the liquid guiding cotton 73 is sleeved on the bulge loop 65 to prevent the liquid atomizable substance in the liquid storage cotton 40 from leaking from the connection between the second oil stopper 60 and the liquid guiding cotton 73. In some embodiments, the air duct 30 may abut the top surface of the liquid guiding cotton 73 to prevent the liquid atomizable substance in the liquid storage cotton 40 from leaking into the air duct 30.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

What is claimed is:

1. An atomizer comprising:
a liquid storage chamber;
an air duct disposed in the liquid storage chamber;
an atomizing core disposed in the liquid storage chamber and comprising a core shell and a liquid guiding cotton, wherein the core shell comprises a base and a housing disposed on the base; and
a sealing member disposed between the air duct and the core shell, wherein the sealing member comprises a sealing body and a first connecting pipe extending from a surface of the sealing body facing the air duct, an elastic part is disposed on an outer wall of the first connecting pipe, the air duct abuts against the sealing body, a bottom of the sealing body abuts against the liquid guiding cotton, the first connecting pipe is located in the air duct, and the elastic part abuts and presses against an inner wall of the air duct.

2. The atomizer of claim 1, wherein the elastic part comprises a plurality of elastic rings.

3. The atomizer of claim 1, wherein an outer wall of the sealing body defines a groove, a portion of the housing is accommodated in the groove.

4. The atomizer of claim 1, wherein the liquid storage chamber comprises a first end and a second end opposite to the first end;
the atomizer further comprises a first oil stopper sealed on the first end and a second connecting pipe extending from a surface of the first oil stopper facing the second end, the first oil stopper comprises a first vent, the second connecting pipe communicates with the first vent, a connecting position between the first oil stopper and the second connecting pipe is recessed to form a recess, an end of the air duct abuts against the recess, and the second connecting pipe extends into the air duct, so that an outer wall of the second connecting pipe abuts against the inner wall of the air duct.

5. The atomizer of claim 4, further comprising a suction part disposed on an end of the liquid storage chamber, wherein a first liquid absorbent cotton is disposed between the suction part and the first oil stopper.

6. The atomizer of claim 4, further comprising a second oil stopper and a liquid storage cotton, wherein the second oil stopper is arranged at the second end, the atomizing core is disposed on the second oil stopper, the liquid storage cotton is disposed in the liquid storage chamber and surrounds outer walls of the air duct and the atomizing core, and the liquid storage cotton is sealed between the first oil stopper and the second oil stopper.

7. The atomizer of claim 6, wherein the second oil stopper comprises a second vent, a surface of the second oil stopper away from the air duct defines a plurality of air passages communicating with the second vent.

8. The atomizer of claim 7, wherein the surface of the second oil stopper away from the air duct further defines a plurality of air grooves, each of the plurality of air grooves communicating with the second vent and one of the plurality of air passages.

9. The atomizer of claim 6, wherein a bulge loop is disposed on a side of the second oil stopper facing the atomizing core, the atomizing core is sleeved on the bulge loop.

10. An inhalation device comprising:
a power supply assembly;
an atomizer comprising:
a liquid storage chamber;
an air duct disposed in the liquid storage chamber;
an atomizing core disposed in the liquid storage chamber and comprising a core shell and a liquid guiding cotton, wherein the core shell comprises a base and a housing disposed on the base; and
a sealing member disposed between the air duct and the core shell, wherein the sealing member comprises a sealing body and a first connecting pipe extending from a surface of the sealing body facing the air duct, an elastic part is disposed on an outer wall of the first connecting pipe, the air duct abuts against the sealing body, a bottom of the sealing body abuts against the liquid guiding cotton, the first connecting pipe is located in the air duct, and the elastic part abuts and presses against an inner wall of the air duct; and
a second liquid absorbent cotton disposed between the power supply assembly and the atomizer and comprising a notch.

11. The inhalation device of claim 10, wherein the elastic part comprises a plurality of elastic rings.

12. The inhalation device of claim 10, wherein an outer wall of the sealing body defines a groove, a portion of the housing is accommodated in the groove.

13. The inhalation device of claim 10, wherein the liquid storage chamber comprises a first end and a second end opposite to the first end;
the atomizer further comprises a first oil stopper sealed on the first end and a second connecting pipe extending from a surface of the first oil stopper facing the second end, the first oil stopper comprises a first vent, the second connecting pipe communicates with the first vent, a connecting position between the first oil stopper and the second connecting pipe is recessed to form a recess, an end of the air duct abuts against the recess, and the second connecting pipe extends into the air duct, so that an outer wall of the second connecting pipe abuts against the inner wall of the air duct.

14. The inhalation device of claim 13, wherein the atomizer further comprises a suction part disposed on an end of the liquid storage chamber, and a first liquid absorbent cotton is disposed between the suction part and the first oil stopper.

15. The inhalation device of claim 13, wherein the atomizer further comprises a second oil stopper and a liquid storage cotton, the second oil stopper is arranged at the second end, the atomizing core is disposed on the second oil stopper, the liquid storage cotton is disposed in the liquid storage chamber and surrounds outer walls of the air duct and the atomizing core, and the liquid storage cotton is sealed between the first oil stopper and the second oil stopper.

16. The inhalation device of claim 15, wherein the second oil stopper comprises a second vent, a surface of the second oil stopper away from the air duct defines a plurality of air passages communicating with the second vent.

17. The inhalation device of claim 16, wherein the surface of the second oil stopper away from the air duct further defines a plurality of air grooves, each of the plurality of air grooves communicating with the second vent and one of the plurality of air passages.

18. The inhalation device of claim 15, wherein a bulge loop is disposed on a side of the second oil stopper facing the atomizing core, the atomizing core is sleeved on the bulge loop.
